(19) United States
(12) Patent Application Publication
(10) Pub. No.: US 2025/0352164 A1
(43) Pub. Date: Nov. 20, 2025

(54) CT APPARATUS AND CONTROL METHOD FOR CT APPARATUS

(71) Applicant: FUJIFILM Corporation, Tokyo (JP)

(72) Inventors: Hideo KONDOH, Tokyo (JP); Takashi ISHIKAWA, Tokyo (JP)

(73) Assignee: FUJIFILM Corporation, Tokyo (JP)

(21) Appl. No.: 19/203,232

(22) Filed: May 9, 2025

(30) Foreign Application Priority Data
May 14, 2024 (JP) 2024-078945

(51) Int. Cl. A61B 6/00; A61B 6/03; A61B 6/42

(52) U.S. Cl. CPC A61B 6/56; A61B 6/032; A61B 6/4241; A61B 6/547

(57) ABSTRACT

A CT apparatus includes: an X-ray source; a detector; a rotating portion; a stationary portion; a transmitting antenna provided on the rotating portion for transmitting detection data; a receiving antenna disposed at a position facing a part of the transmitting antenna; a rotational position detector configured to detect a rotational position of the rotating portion and output a detection value; a measurement sensor configured to measure a relative relationship between the transmitting antenna and the receiving antenna and output a measurement value; a displacement mechanism configured to displace at least one of the receiving antenna or the transmitting antenna; and a processor configured to create control data based on the detection value and the measurement value, and execute displacement control to suppress a change in the relative relationship by controlling the displacement mechanism based on the control data during transmission and reception of the detection data.

### CROSS-REFERENCE TO RELATED APPLICATION

The present application claims priority under 35 U.S.C. § 119 to Japanese Patent Application No. 2024-078945, filed on May 14, 2024. The above application is hereby expressly incorporated by reference, in its entirety, into the present application.

### BACKGROUND

### 1. Technical Field

The present disclosure relates to a CT apparatus and a control method for a CT apparatus.

### 2. Description of the Related Art

A computed tomography (CT) apparatus irradiates a subject with X-rays while rotating a rotating portion, in which an X-ray source and a detector are disposed in positions facing each other, around the subject, and detects the X-rays transmitted through the subject with the detector. Detection data detected by the detector is transmitted from the rotating portion to a stationary portion that rotatably holds the rotating portion, and image processing such as reconstruction processing is performed by a console or the like connected to the stationary portion.

In order to transmit the detection data from the rotating portion to the stationary portion, a non-contact data transmission device is used (see, for example, JP2013-244148A). As non-contact transmission methods, a capacitive coupling method using capacitive coupling and an optical transmission method using light are known.

### SUMMARY

In recent years, the capacity of data transmitted from a rotating portion to a stationary portion has been increasing. For example, in a photon counting computed tomography (PCCT) apparatus that uses a photon counting detector that counts incident X-ray photons, the capacity of detected data is large, and thus it is necessary to transmit a large capacity of data at high speed from the rotating portion to the stationary portion.

In a case in which a capacitively coupled data transmission device is applied to a CT apparatus, a transmitting antenna is provided on the rotating portion, and a receiving antenna is provided on the stationary portion. In this case, it is necessary to dispose the transmitting antenna and the receiving antenna such that they are reliably capacitively coupled. For example, the transmitting antenna extends along the outer peripheral portion or the inner peripheral portion of the rotating portion, and the receiving antenna is disposed to face a part of the transmitting antenna.

In the capacitive coupling method, the transmission band is proportional to the coupling capacitance between the transmitting antenna and the receiving antenna. The coupling capacitance depends on the distance between the transmitting antenna and the receiving antenna and the area where the transmitting antenna and the receiving antenna overlap. In order to perform stable data transmission using the capacitive coupling method, it is necessary to maintain a constant positional relationship between the transmitting antenna and the receiving antenna in a state in which the rotating portion is rotating.

However, the rotating portion may be deformed due to deterioration over time or the like. In a case in which the rotating portion is deformed, the relative relationship between the transmitting antenna and the receiving antenna changes as the rotating portion rotates, causing a change in coupling capacitance, which reduces the stability of data transmission. In particular, in a case in which the distance between the transmitting antenna and the receiving antenna is reduced in order to increase the transmission speed, the deformation of the rotating portion has a large effect.

For example, in order to improve the stability of data transmission, it is conceivable to measure the distance between a transmitting antenna and a receiving antenna and adjust the position of the receiving antenna in accordance with the measurement value. However, it is not easy to displace the receiving antenna based on the measurement value while measuring the distance in a state in which the rotating portion is rotating. For example, it is considered that it may take time for the receiving antenna to displace and it may not be able to follow the rotation of the rotating portion, which may reduce the stability of data transmission.

Therefore, an object of the technology according to the present disclosure is to provide a CT apparatus and a control method for a CT apparatus that make it possible to improve the stability of data transmission using a capacitive coupling method.

According to the technology of the present disclosure, there is provided a CT apparatus comprising: an X-ray source configured to radiate X-rays to a subject; a detector configured to detect X-rays transmitted through the subject and output detection data; a rotating portion configured to support the X-ray source and the detector and rotate around a rotation axis; a stationary portion configured to rotatably hold the rotating portion; a transmitting antenna provided on the rotating portion for transmitting the detection data; a receiving antenna disposed at a position facing a part of the transmitting antenna; a rotational position detection unit configured to detect a rotational position of the rotating portion and output a detection value; a measurement unit configured to measure a relative relationship between the transmitting antenna and the receiving antenna and output a measurement value; a displacement mechanism configured to displace at least one of the receiving antenna or the transmitting antenna; and a processor configured to create control data based on the detection value and the measurement value, and execute displacement control to suppress a change in the relative relationship by controlling the displacement mechanism based on the control data during transmission and reception of the detection data.

It is preferable that the processor is configured to create relative relationship data indicating a relationship between the relative relationship and the rotational position for one period based on the detection value and the measurement value for one period of rotation of the rotating portion, and create the control data based on the relative relationship data.

It is preferable that the control data indicates a relationship between a displacement amount of at least one of the receiving antenna or the transmitting antenna for one period and the rotational position.

It is preferable that the transmitting antenna is disposed along an outer peripheral portion or an inner peripheral portion of the rotating portion.

It is preferable that the relative relationship is a distance between the transmitting antenna and the receiving antenna, and the displacement mechanism is configured to displace the receiving antenna in a direction parallel to the rotation axis.

The relative relationship may be a parallelism between the transmitting antenna and the receiving antenna, and the displacement mechanism may be configured to displace the receiving antenna around an axis parallel to the rotation axis.

The relative relationship may be an overlap rate between the transmitting antenna and the receiving antenna, and the displacement mechanism may be configured to displace the receiving antenna around an axis parallel to a direction orthogonal to the rotation axis.

It is preferable that the CT apparatus further comprises a receiver configured to receive the detection data via the receiving antenna, and the displacement mechanism is configured to displace the receiving antenna by displacing the receiver.

The processor may be configured to correct the control data based on the measurement value during execution of the displacement control.

The processor may be configured to correct the control data based on a difference value between the measurement value before one period, before a certain time, or before a certain rotation angle and the acquired measurement value.

According to the technology of the present disclosure, there is provided a control method for a CT apparatus including an X-ray source configured to radiate X-rays to a subject, a detector configured to detect X-rays transmitted through the subject and output detection data, a rotating portion configured to support the X-ray source and the detector and rotate around a rotation axis, a stationary portion configured to rotatably hold the rotating portion, a transmitting antenna provided on the rotating portion for transmitting the detection data, a receiving antenna disposed at a position facing a part of the transmitting antenna, a rotational position detection unit configured to detect a rotational position of the rotating portion and output a detection value, a measurement unit configured to measure a relative relationship between the transmitting antenna and the receiving antenna and output a measurement value, and a displacement mechanism configured to displace at least one of the receiving antenna or the transmitting antenna, the control method comprising: creating, by a processor, control data based on the detection value and the measurement value; and executing displacement control to suppress a change in the relative relationship by controlling the displacement mechanism based on the control data during transmission and reception of the detection data.

According to the technology of the present disclosure, it is possible to provide a CT apparatus and a control method for a CT apparatus that make it possible to improve the stability of data transmission using a capacitive coupling method.

### BRIEF DESCRIPTION OF THE DRAWINGS

Exemplary embodiments according to the technique of the present disclosure will be described in detail based on the following figures, wherein:

FIG. 1 is a diagram schematically showing a configuration of a CT apparatus,

FIG. 2 is a diagram schematically showing a configuration of a gantry,

FIG. 3 is a diagram showing a configuration of a data transmission device,

FIG. 4 is a diagram schematically showing an example of a configuration of a transmitting antenna and a receiving antenna,

FIG. 5 is a diagram schematically showing an example of a configuration of a transmitting antenna and a receiving antenna,

FIG. 6 is a diagram showing an example of a configuration of a displacement controller,

FIG. 7 is a diagram showing an example of a flow of a control data creation process,

FIG. 8 is a diagram showing an example of relative relationship data,

FIG. 9 is a diagram showing an example of control data,

FIG. 10 is a diagram showing an example of a flow of displacement control,

FIG. 11 is a diagram showing a configuration of a measurement unit and a displacement mechanism according to a first modification example,

FIG. 12 is a diagram illustrating parallelism,

FIG. 13 is a diagram illustrating an overlap rate,

FIG. 14 is a diagram showing an example of a flow of displacement control according to a second modification example, and

FIG. 15 is a diagram schematically showing an example of a configuration of a transmitting antenna and a receiving antenna according to a third modification example.

### DETAILED DESCRIPTION

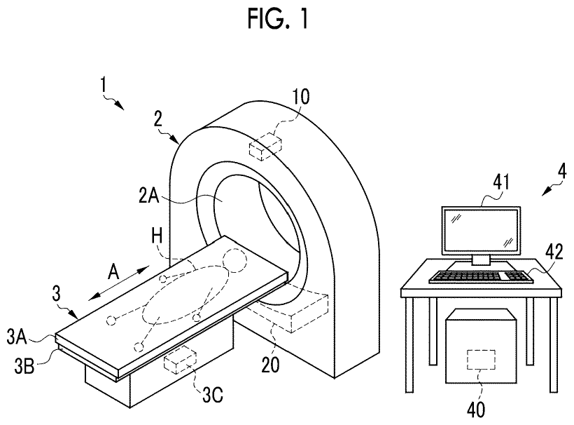

FIG. 1 schematically shows the configuration of a CT apparatus 1. The CT apparatus 1 is composed of a gantry 2, a bed 3, and a console 4. The CT apparatus 1 is not limited to a CT apparatus having a charge integration type detector, but may be a PCCT apparatus having a photon counting detector that counts the photons of incident X-rays.

The gantry 2 has an opening 2A in the center through which a part of the bed 3 is inserted. An X-ray source 10 that radiates X-rays to a subject H and a detector 20 that detects the X-rays transmitted through the subject H to generate a radiation image are provided inside the gantry 2. The X-ray source 10 and the detector 20 are configured to be rotatable along the annular shape of the gantry 2 while maintaining a mutually opposing positional relationship.

The bed 3 has a top plate 3A on which the subject H is placed, a base 3B that supports the top plate 3A, and a driving unit 3C that moves the top plate 3A back and forth in the direction of arrow A, and is configured to allow the subject H to be moved. The top plate 3A can be slid in the direction of arrow A relative to the base 3B by the driving unit 3C. In a case in which the subject H is imaged, the top plate 3A slides such that the top plate 3A is inserted into the opening 2A of the gantry 2. Accordingly, the subject H is transported into the opening 2A.

The console 4 is a computer including a processor 40 such as a central processing unit (CPU), a display 41 such as a liquid-crystal display, and an input device 42 such as a keyboard and a mouse.

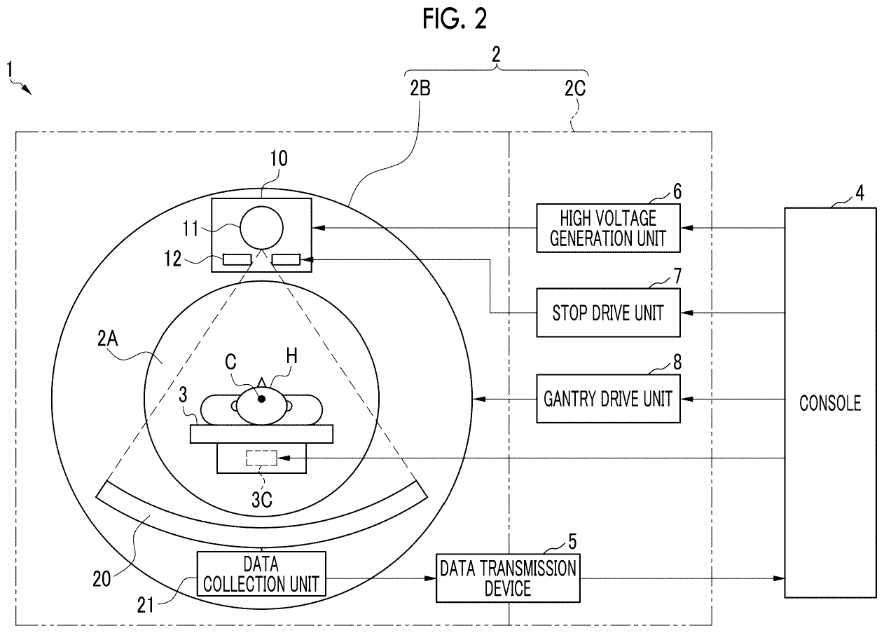

FIG. 2 schematically shows the configuration of the gantry 2. The gantry 2 is a device that irradiates the subject H with X-rays and collects detection data of the X-rays transmitted through the subject H. The gantry 2 has a rotating portion 2B and a stationary portion 2C. A data transmission device 5 is provided between the rotating portion 2B and the stationary portion 2C for transmitting data from the rotating portion 2B to the stationary portion 2C in a non-contact manner.

The rotating portion 2B supports the X-ray source 10, the detector 20, and the data collection unit 21. The rotating portion 2B is a disk-shaped rotating body that supports the X-ray source 10 and the detector 20 such that they can rotate about a rotation axis C in a state in which they face each other. The above-mentioned opening 2A is formed in the center of the rotating portion 2B. The data collection unit 21 is attached to the detector 20. The rotating portion 2B rotates in a circular orbit centered on the rotation axis C. The subject H is disposed such that the body axis is substantially aligned with the rotation axis C.

The X-ray source 10 includes an X-ray tube 11 and a stop 12. The X-ray tube 11 generates X-rays and irradiates the subject H with the generated X-rays. The stop 12 shapes the X-rays generated by the X-ray tube 11 into a cone beam having a predetermined fan angle and cone angle.

The detector 20 includes a plurality of X-ray detection elements. The detector 20 detects data indicating the intensity distribution of X-rays transmitted through the subject H (hereinafter referred to as “detection data”) using a plurality of X-ray detection elements, and outputs the detection data. For example, the detector 20 is a two-dimensional X-ray detector in which a plurality of X-ray detection elements are disposed in two mutually orthogonal directions (that is, a slice direction and a channel direction). The detector 20 can image a three-dimensional imaging region having a width in the slice direction in one rotation scan. The slice direction is a direction parallel to the rotation axis C, and the channel direction is a rotation direction centered on the rotation axis C.

The data collection unit 21 is a data acquisition system (DAS) that collects detection data output from the detector 20. In addition, the data collection unit 21 converts the collected detection data into digital data and transmits it to the data transmission device 5.

The stationary portion 2C is a holding member that rotatably holds the rotating portion 2B. The stationary portion 2C has a high voltage generation unit 6, a stop drive unit 7, and a gantry drive unit 8.

The high voltage generation unit 6 applies a high voltage to the X-ray tube 11 to cause the X-ray tube 11 to generate X-rays. The stop drive unit 7 drives the stop 12 such that the X-rays generated by the X-ray tube 11 have a predetermined shape. The gantry drive unit 8 drives the rotating portion 2B to rotate.

The high voltage generation unit 6, the stop drive unit 7, and the gantry drive unit 8 are controlled by the console 4. The console 4 also controls the driving unit 3C of the bed 3.

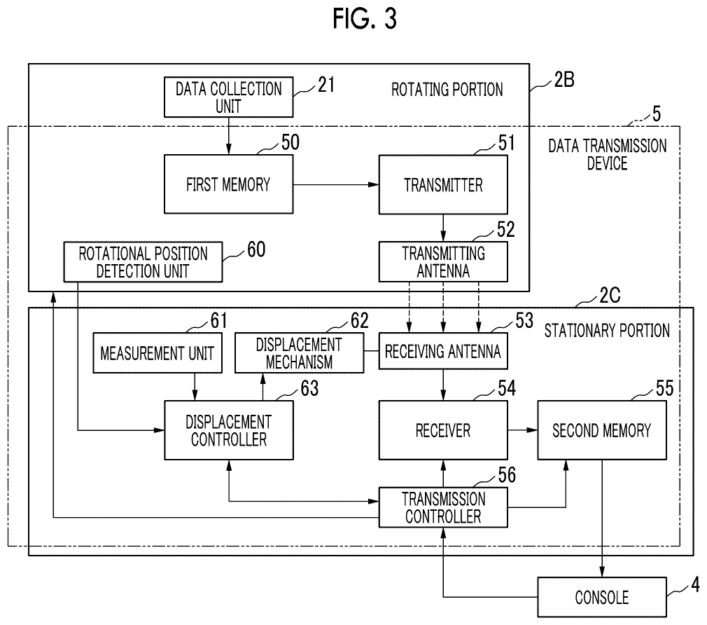

FIG. 3 shows the configuration of the data transmission device 5. The data transmission device 5 includes a first memory 50, a transmitter 51, a transmitting antenna 52, a receiving antenna 53, a receiver 54, a second memory 55, and a transmission controller 56. The first memory 50, the transmitter 51, and the transmitting antenna 52 are provided on the rotating portion 2B. The receiving antenna 53, the receiver 54, the second memory 55, and the transmission controller 56 are provided in the stationary portion 2C.

The first memory 50 stores the detection data collected by the data collection unit 21. The transmitter 51 is a transmitting circuit that transmits the detection data stored in the first memory 50 via the transmitting antenna 52. Specifically, the transmitter 51 generates a high-frequency electric signal by modulating the amplitude, phase, frequency, and the like of a carrier wave based on the detection data, and supplies the electric signal to the transmitting antenna 52. The transmitting antenna 52 emits the electric signal supplied from the transmitter 51 as a radio wave.

The receiver 54 is a receiving circuit that receives, via the receiving antenna 53, the detection data transmitted from the transmitter 51 via the transmitting antenna 52, and stores the data in the second memory 55. Specifically, the receiving antenna 53 detects the radio waves emitted from the transmitting antenna 52 to generate an electric signal, and supplies the generated electric signal to the receiver 54. The receiver 54 demodulates the electric signal supplied from the receiving antenna 53 to convert the electric signal into detection data, and stores the detection data in the second memory 55.

The transmission controller 56 controls each of the first memory 50, the transmitter 51, the transmitting antenna 52, the receiving antenna 53, the receiver 54, and the second memory 55 to transmit and receive detection data (hereinafter also referred to as data transmission). In addition, the transmission controller 56 causes the detection data to be transmitted from the second memory 55 to the console 4.

The data transmission device 5 also has a rotational position detection unit 60, a measurement unit 61, a displacement mechanism 62, and a displacement controller 63. For example, the rotational position detection unit 60 is provided in the rotating portion 2B, and the measurement unit 61, the displacement mechanism 62, and the displacement controller 63 are provided in the stationary portion 2C. The rotational position detection unit 60 corresponds to a rotational position detector in the present disclosure.

The rotational position detection unit 60 detects the rotational position of the rotating portion 2B. The rotational position detection unit 60 is, for example, an optical, magnetic, or mechanical rotary encoder. The rotational position is the rotation angle from a reference angle. The detection value of the rotational position detected by the rotational position detection unit 60 is transmitted to the displacement controller 63 via wire or wirelessly.

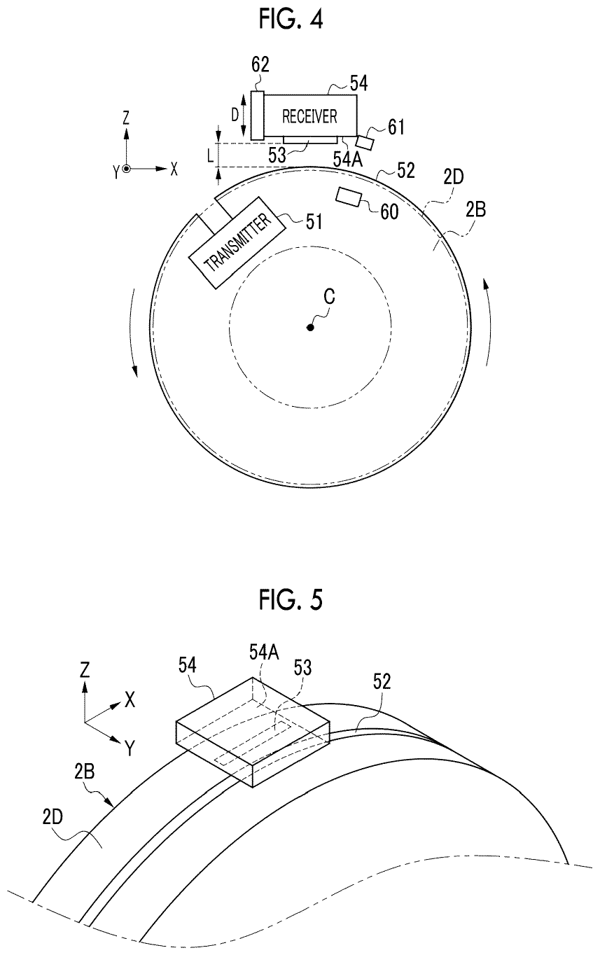

The measurement unit 61 is a measurement sensor that measures the relative relationship between the transmitting antenna 52 and the receiving antenna 53 and supplies the measurement value to the displacement controller 63. In the present embodiment, the measurement unit 61 measures a distance L (see FIG. 4) between the transmitting antenna 52 and the receiving antenna 53. For example, the measurement unit 61 is a laser displacement meter, and is fixed near the receiving antenna 53. The measurement unit 61 measures the displacement of the transmitting antenna 52 to measure the distance L between the transmitting antenna 52 and the receiving antenna 53.

The measurement unit 61 may be any device capable of measuring the relative relationship between the transmitting antenna 52 and the receiving antenna 53, and may be configured with an optical device such as a dial gauge or a camera. The measurement unit 61 may also be a gyro sensor provided inside the rotating portion 2B. Furthermore, it is also possible to measure the distance L between the transmitting antenna 52 and the receiving antenna 53 based on the intensity of the radio waves emitted from the transmitting antenna 52 detected by the receiving antenna 53.

The displacement mechanism 62 is a mechanism for changing the relative position of the receiving antenna 53 with respect to the transmitting antenna 52. The receiving antenna 53 is fixed to the receiver 54. For this reason, the displacement mechanism 62 is provided in the receiver 54, and displaces the receiver 54 to displace the receiving antenna 53 relative to the transmitting antenna 52. In the present disclosure, displacement includes not only translational movement of an object but also rotational movement.

In the present embodiment, the displacement mechanism 62 displaces (that is, translates) the receiving antenna 53 such that the distance L between the transmitting antenna 52 and the receiving antenna 53 changes. For example, the displacement mechanism 62 is an actuator having a piezoelectric motor, a stepping motor, a servo motor, or the like.

The displacement controller 63 performs displacement control to suppress changes in the relative relationship between the transmitting antenna 52 and the receiving antenna 53 by controlling the displacement mechanism 62 in a state in which the rotating portion 2B is rotating. In the present embodiment, the displacement controller 63 controls the displacement mechanism 62 to displace the receiving antenna 53 in the radial direction of the rotating portion 2B (a Z direction shown in FIG. 3), thereby suppressing the change in the distance L between the transmitting antenna 52 and the receiving antenna 53 during rotation.

In addition, before performing displacement control, the displacement controller 63 acquires the detection value of the rotational position detected by the rotational position detection unit 60 in a state in which the rotating portion 2B is rotating, and the measurement value of the relative relationship between the transmitting antenna 52 and the receiving antenna 53 measured by the measurement unit 61. The displacement controller 63 creates control data for canceling changes in the relative relationship between the transmitting antenna 52 and the receiving antenna 53 based on the acquired data indicating the relationship between the rotational position and the relative relationship. Then, the displacement controller 63 performs displacement control using the created control data.

FIG. 4 schematically shows an example of the configuration of the transmitting antenna 52 and the receiving antenna 53. The transmitting antenna 52 is composed of a conductive member disposed along an outer peripheral portion 2D of the rotating portion 2B, and both ends of the conductive member are connected to the transmitter 51. That is, the transmitting antenna 52 is a part of a transmitting circuit (not shown) and has a pattern shape such as a circular shape, an arc shape, or a meandering shape disposed along the outer peripheral portion 2D centered on the rotation axis C. The outer peripheral portion 2D refers to the outer portion of the rotating portion 2B. In the present embodiment, the outer peripheral portion 2D is an outer peripheral surface centered on the rotation axis C, and the transmitting antenna 52 is disposed on the outer peripheral surface and exposed.

The receiving antenna 53 is disposed at a position facing a part of the transmitting antenna 52 disposed along the outer peripheral portion 2D of the rotating portion 2B. The receiving antenna 53 is composed of a conductive member disposed on a surface 54A of the receiver 54. As shown in FIG. 5, the receiving antenna 53 is a part of a receiving circuit (not shown), faces a part of the transmitting antenna 52, and is disposed in parallel to the transmitting antenna 52 in a pattern shape such as a linear shape, an arc shape, or a meandering shape.

In FIGS. 4 and 5, the direction parallel to the rotation axis C is defined as a Y direction, the direction orthogonal to the Y direction in which the transmitting antenna 52 and the receiving antenna 53 face each other is defined as a Z direction, and the direction orthogonal to the Y direction and the Z direction is defined as an X direction. The receiving antenna 53 is parallel to the X direction. In addition, a tangent line orthogonal to the rotation axis C at a portion of the transmitting antenna 52 facing the receiving antenna 53 is substantially parallel to the X direction.

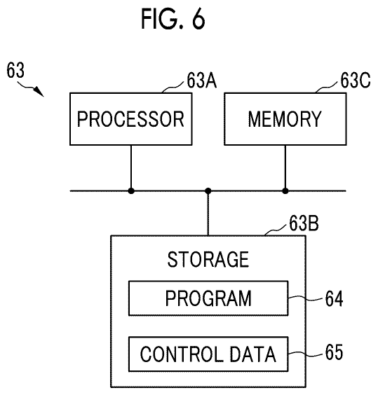

FIG. 6 shows an example of the configuration of the displacement controller 63. The displacement controller 63 includes a processor 63A such as a central processing unit (CPU), a non-volatile storage 63B, and a memory 63C as a temporary storage area. The non-volatile storage 63B stores a program 64 and control data 65.

The displacement controller 63 executes a control data creation process of creating the control data 65 and the above-mentioned displacement control by executing processing based on the program 64 read by the processor 63A into the memory 63C.

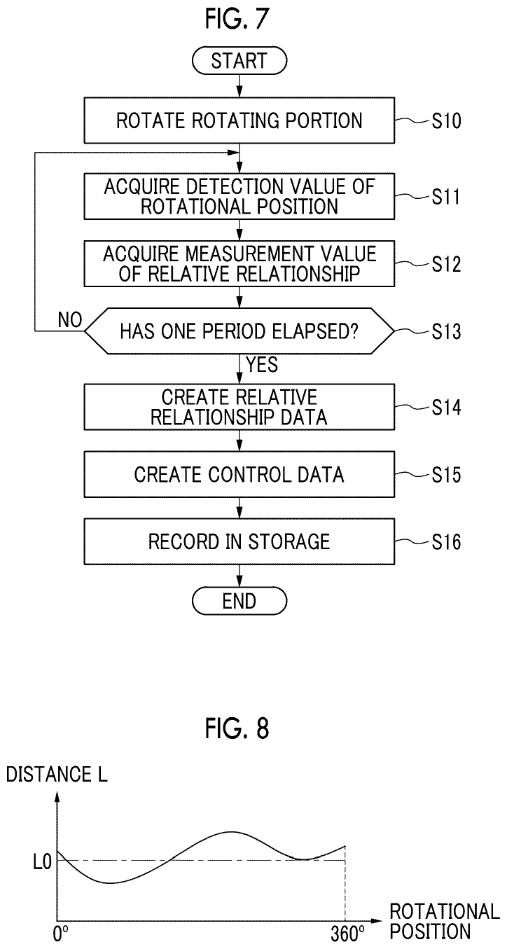

FIG. 7 shows an example of the flow of a control data creation process. In the control data creation process, first, the displacement controller 63 rotates the rotating portion 2B via the console 4 (Step S10). Next, the displacement controller 63 acquires a detection value of the rotational position from the rotational position detection unit 60 (Step S11). In addition, the displacement controller 63 acquires a measurement value of the relative relationship between the transmitting antenna 52 and the receiving antenna 53 from the measurement unit 61 (Step S12). Note that Steps S11 and S12 may be executed in parallel.

Next, the displacement controller 63 determines whether or not one period of rotation of the rotating portion 2B has elapsed (Step S13). In a case in which one period of rotation of the rotating portion 2B has not elapsed (Step S13: NO), the displacement controller 63 returns the process to Step S11. That is, the displacement controller 63 executes Steps S11 and S12 at predetermined time intervals until one period of rotation of the rotating portion 2B has elapsed.

In a case in which one period of rotation of the rotating portion 2B has elapsed (Step S13: YES), the displacement controller 63 creates relative relationship data indicating the relationship between the relative relationship for one period and the rotational position based on the detection value of the rotational position for one period and the measurement value of the relative relationship (Step S14). Then, the displacement controller 63 creates control data 65 for canceling the change in the relative relationship based on the relative relationship data (Step S15), and records the created control data 65 in the storage 63B (Step S16). As described above, the control data creation process ends.

The displacement controller 63 may create the relative relationship data by performing an averaging process or the like on data of the detection values of the rotational positions and the measurement values of the relative relationship for two or more periods.

Moreover, it is preferable that the displacement controller 63 performs the control data creation process in a state in which the subject H is not disposed on the bed 3. For example, the displacement controller 63 may perform the control data creation process during warm-up after the CT apparatus 1 is activated, during preparation for imaging, or the like. Furthermore, the control data creation process may be performed in a state in which the subject H is disposed on the bed 3. For example, the displacement controller 63 may perform the control data creation process at one or more timings while the CT apparatus 1 is imaging the subject H.

FIG. 8 shows an example of the relative relationship data. The relative relationship data shown in FIG. 8 indicates the relationship between the distance L between the transmitting antenna 52 and the receiving antenna 53 and the rotational position. LO is the ideal distance for stable data transmission. In a case in which the rotating portion 2B is deformed due to deterioration over time or the like, the distance L between the transmitting antenna 52 and the receiving antenna 53 changes with respect to the ideal distance LO.

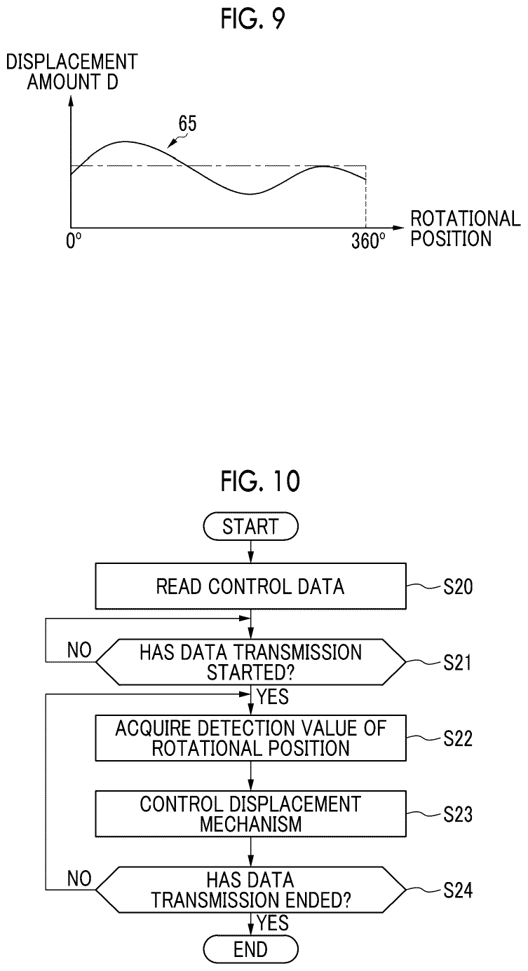

FIG. 9 shows an example of the control data 65. The control data 65 shown in FIG. 9 indicates the relationship between a displacement amount D of the receiving antenna 53 displaced by the displacement mechanism 62 and the rotational position. The displacement amount D is the amount of drive of the receiving antenna 53 in the Z direction required to suppress the change in the distance L between the transmitting antenna 52 and the receiving antenna 53 and make the distance L the ideal distance L 0.

FIG. 10 shows an example of the flow of the displacement control. In the displacement control, first, the displacement controller 63 reads the control data 65 from the storage 63B to the memory 63C (Step S20). Next, the displacement controller 63 determines whether or not the data transmission device 5 has started transmitting the detection data (Step S21). In a case in which data transmission has not started (Step S21: NO), the displacement controller 63 repeats the determination.

In a case in which data transmission has started (Step S21: YES), the displacement controller 63 acquires a detection value of the rotational position from the rotational position detection unit 60 (Step S22). Furthermore, the displacement controller 63 obtains a displacement amount D corresponding to the rotational position based on the control data 65, and controls the displacement mechanism 62 based on the displacement amount D (Step S23).

The displacement controller 63 determines whether or not the data transmission has ended (Step S24). In a case in which the data transmission has not ended (Step S24: NO), the displacement controller 63 returns the process to Step S22. That is, the displacement controller 63 executes Steps S22 and S23 at predetermined time intervals until the data transmission ends. In a case in which the data transmission has ended (Step S24: YES), the displacement controller 63 ends the displacement control.

As described above, in the present embodiment, the receiving antenna 53 is displaced to suppress changes in the distance L between the transmitting antenna 52 and the receiving antenna 53 based on the control data 65 created in advance, thereby stabilizing the coupling capacitance between the transmitting antenna 52 and the receiving antenna 53. In the present embodiment, since the control data 65 created in advance is used, there is no need to perform a calculation to obtain the displacement amount D during rotation, and the ability to follow the displacement of the receiving antenna 53 in response to the rotation of the rotating portion 2B is improved. Therefore, according to the present embodiment, it is possible to improve the stability of data transmission using the capacitive coupling method.

Various modification examples of the above embodiment will be described below.

### First Modification Example

In the above embodiment, the measurement unit 61 measures the distance L between the transmitting antenna 52 and the receiving antenna 53 as the relative relationship between the transmitting antenna 52 and the receiving antenna 53, but a relative relationship other than the distance L may also be measured. In the above embodiment, the displacement mechanism 62 displaces the receiving antenna 53 in the radial direction of the rotating portion 2B (that is, the Z direction), but the displacement mechanism 62 may displace the receiving antenna 53 in a direction other than the Z direction.

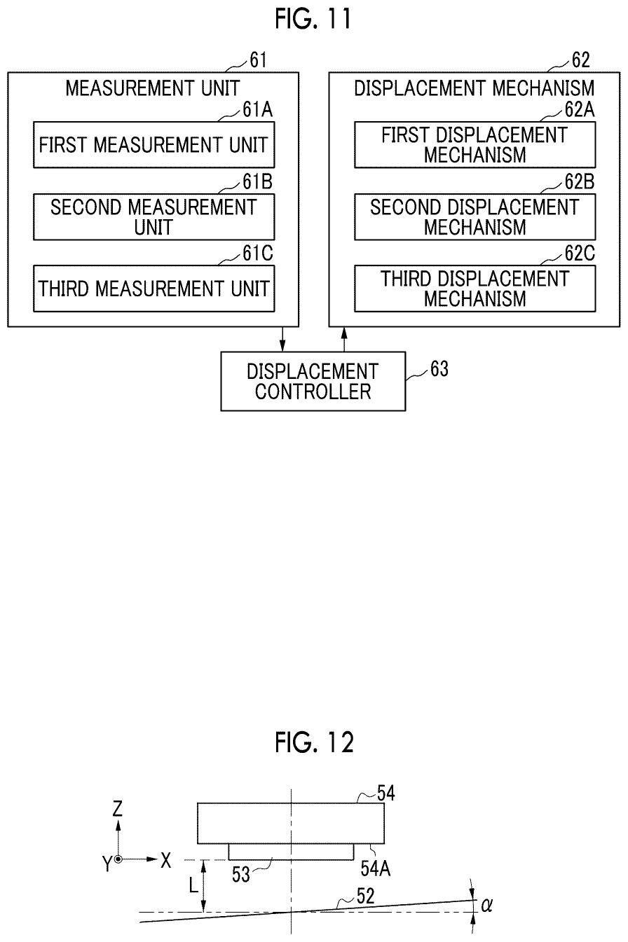

FIG. 11 shows the configuration of a measurement unit 61 and a displacement mechanism 62 according to a first modification example. In the present modification example, the measurement unit 61 includes a first measurement unit 61A, a second measurement unit 61B, and a third measurement unit 61C. The displacement mechanism 62 includes a first displacement mechanism 62A, a second displacement mechanism 62B, and a third displacement mechanism 62C.

The first measurement unit 61A has a configuration similar to that of the measurement unit 61 in the above embodiment, and measures the distance L between the transmitting antenna 52 and the receiving antenna 53.

The second measurement unit 61B measures the parallelism between the transmitting antenna 52 and the receiving antenna 53. As shown in FIG. 12, the portion of the transmitting antenna 52 facing the receiving antenna 53 is substantially flat. Specifically, an inclined angle α of the portion of the transmitting antenna 52 facing the receiving antenna 53 with respect to the transmitting antenna 52 is measured. The inclined angle α is an angle around an axis parallel to the Y direction. The smaller the inclined angle α, the higher the parallelism and the more stable the data transmission.

For example, the second measurement unit 61B is a plurality of laser displacement meters disposed in the X direction. The second measurement unit 61B measures the distance between the transmitting antenna 52 and the receiving antenna 53 at a plurality of positions in the X direction, thereby measuring the inclined angle α. The second measurement unit 61B may be a camera that captures images of the transmitting antenna 52 and the receiving antenna 53 from the Y direction, and may measure the inclined angle α based on the captured image.

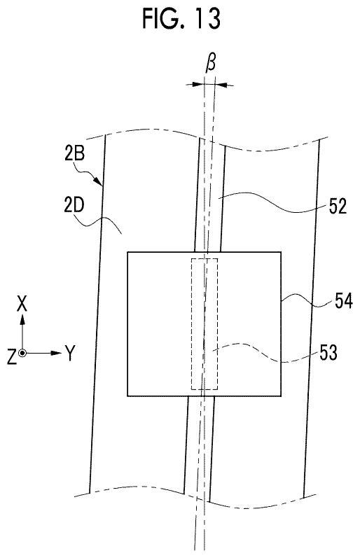

The third measurement unit 61C measures the overlap rate between the transmitting antenna 52 and the receiving antenna 53. As shown in FIG. 13, in a case of being viewed from the Z direction, the transmitting antenna 52 and the receiving antenna 53 substantially overlap, but in a case in which the transmitting antenna 52 is inclined in the XY plane, the overlap rate decreases. The third measurement unit 61C measures an inclined angle β of the portion of the transmitting antenna 52 facing the receiving antenna 53 with respect to the transmitting antenna 52. The inclined angle β is an angle around an axis parallel to the Z direction. The smaller the inclined angle β, the higher the overlap rate and the more stable the data transmission.

For example, the third measurement unit 61C is a camera that captures an image of the transmitting antenna 52 from the Z direction. The third measurement unit 61C measures the inclined angle β based on the captured image.

The first displacement mechanism 62A has a configuration similar to that of the displacement mechanism 62 in the above embodiment, and displaces the receiving antenna 53 in the Z direction such that the distance L between the transmitting antenna 52 and the receiving antenna 53 changes.

The second displacement mechanism 62B displaces (that is, rotates) the receiving antenna 53 around an axis parallel to the Y axis to change the inclined angle α. For example, the second displacement mechanism 62B is a rotary actuator. For example, the second displacement mechanism 62B is fixed to the receiver 54, and displaces the receiving antenna 53 by displacing the receiver 54.

The third displacement mechanism 62C displaces (that is, rotates) the receiving antenna 53 around an axis parallel to the Z axis to change the inclined angle β. For example, the third displacement mechanism 62C is a rotary actuator. For example, the third displacement mechanism 62C is fixed to the receiver 54, and displaces the receiving antenna 53 by displacing the receiver 54.

In the present modification example, the displacement controller 63 performs a first displacement control, a second displacement control, and a third displacement control independently in a state in which the rotating portion 2B is rotating. The first displacement control is a displacement control similar to that of the above embodiment, which controls the first displacement mechanism 62A based on the detection value of the rotational position detected by the rotational position detection unit 60 and the measurement value measured by the first measurement unit 61A.

The second displacement control is a displacement control that controls the second displacement mechanism 62B based on the detection value of the rotational position detected by the rotational position detection unit 60 and the measurement value measured by the second measurement unit 61B. The third displacement control is a displacement control that controls the third displacement mechanism 62C based on the detection value of the rotational position detected by the rotational position detection unit 60 and the measurement value measured by the third measurement unit 61C.

As in the above embodiment, the displacement controller 63 creates first relative relationship data, second relative relationship data, and third relative relationship data before executing the first displacement control, the second displacement control, and the third displacement control. The first relative relationship data is the same relative relationship data as that in the above embodiment, which indicates the relationship between the distance L between the transmitting antenna 52 and the receiving antenna 53 and the rotational position.

The second relative relationship data is relative relationship data that indicates the relationship between the parallelism (that is, the inclined angle α) and the rotational position. The third relative relationship data is relative relationship data that indicates the relationship between the overlap rate (that is, the inclined angle β) and the rotational position.

Furthermore, the displacement controller 63 performs a control data creation process of creating first control data, second control data, and third control data based on the first relative relationship data, the second relative relationship data, and the third relative relationship data. The first control data indicates the relationship between the displacement amount D of the receiving antenna 53 in the Z direction displaced by the first displacement mechanism 62A and the rotational position, as in the above embodiment.

The second control data indicates the relationship between the displacement amount (that is, the rotation amount) of the receiving antenna 53 around an axis parallel to the Y axis displaced by the second displacement mechanism 62B and the rotational position. The third control data indicates the relationship between the displacement amount (that is, the rotation amount) of the receiving antenna 53 around an axis parallel to the Z axis displaced by the third displacement mechanism 62C and the rotational position.

The first control data, the second control data, and the third control data each indicate the relationship between the rotational position and the displacement amount of the rotating portion 2B for one period. The displacement controller 63 may create the first relative relationship data, the second relative relationship data, and the third relative relationship data by performing an averaging process or the like on data of the detection values of the rotational positions and the measurement values of the relative relationship for two or more periods.

The execution timing of the control data creation process in the present modification example is the same as in the above embodiment.

The technology of the present disclosure may be configured to execute one or more of the first displacement control, the second displacement control, and the third displacement control.

### Second Modification Example

In the above embodiment, the displacement controller 63 performs displacement control based on the control data 65 created in advance, but the control data 65 may be corrected based on the measurement value measured by the measurement unit 61 while the displacement control is being performed. After the control data 65 is created, there is a concern that the control data 65 will deviate from the optimum value due to changes in environmental conditions such as temperature, humidity, and air pressure, and changes in a device status such as the rotation speed of the rotating portion 2B, the scan time, and the number of years the device has been used. This may result in a decrease in the stability of data transmission. By correcting the control data 65 based on the measurement values while displacement control is being performed as in the present modification example, the effects of changes in the environmental conditions or device status described above are suppressed, and the stability of data transmission is improved.

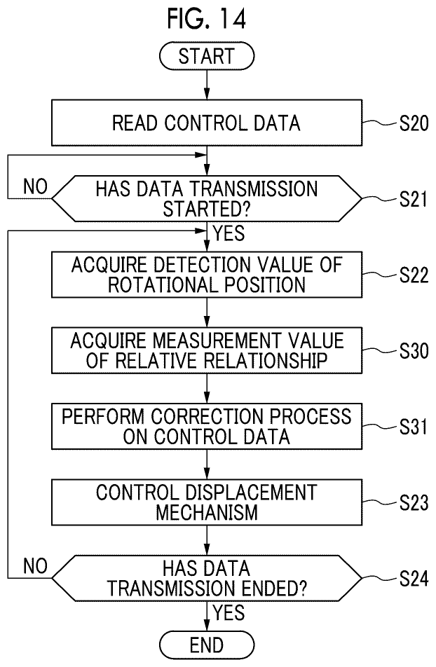

FIG. 14 shows an example of the flow of displacement control according to a second modification example. The flow of displacement control according to the present modification example differs from the flow of displacement control according to the above embodiment only in that Steps S30 and S31 are added between Steps S22 and S23.

In the present modification example, the displacement controller 63 acquires a detection value of the rotational position from the rotational position detection unit 60 in Step S22, and then acquires a measurement value of the relative relationship between the transmitting antenna 52 and the receiving antenna 53 from the measurement unit 61 (Step S30). Next, the displacement controller 63 corrects the control data 65 based on the acquired measurement values (Step S31). Specifically, the displacement controller 63 corrects the displacement amount corresponding to the rotational position acquired in Step S22 based on a difference value between the measurement value before one period, before a certain time, or before a certain rotation angle and the measurement value acquired in Step S30. For example, the displacement controller 63 performs correction with a displacement amount (that is, a control amount) calculated by proportional-integral-differential (PID) control, moving average, feedback control, feedforward control, or the like.

Thereafter, the displacement controller 63 controls the displacement mechanism 62 based on the displacement amount corrected in Step S31 (Step S23).

The second modification example may be applied to each of the first displacement control, the second displacement control, and the third displacement control described in the first modification example above.

The technology of the present disclosure uses the above-mentioned displacement control to maintain the relative relationship between the transmitting antenna 52 and the receiving antenna 53 at a constant value. This constant value includes an allowable error. It is preferable that this allowable error is changed according to, for example, the transmission band of data transmission. In a case in which the transmission speed is high, it is preferable to maintain the relative relationship between the transmitting antenna 52 and the receiving antenna 53 at a constant value with high accuracy, and therefore it is preferable to make the allowable error smaller than in a case in which the transmission speed is low.

### Third Modification Example

In the above embodiment, the transmitting antenna 52 is disposed to be exposed to the outside of the rotating portion 2B, but the transmitting antenna 52 may be disposed inside the rotating portion 2B.

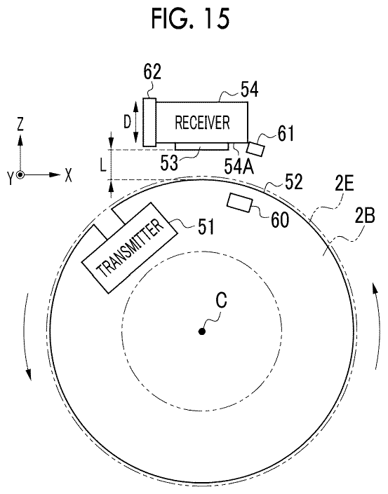

FIG. 15 schematically shows an example of the configuration of a transmitting antenna 52 and a receiving antenna 53 according to a third modification example. In the present modification example, the transmitting antenna 52 is a part of a transmitting circuit (not shown) and has a pattern shape such as a circular shape, an arc shape, or a meandering shape disposed along an inner peripheral portion 2E centered on the rotation axis C. The inner peripheral portion 2E refers to a portion located inside the outer peripheral surface of the rotating portion 2B. In the present modification example, the inner peripheral portion 2E is a region in the vicinity of the outer peripheral surface centered on the rotation axis C, and the transmitting antenna 52 is not exposed from the outer peripheral surface. The configuration of the receiving antenna 53 is the same as in the above embodiment.

### Other Modification Examples

In the above embodiment, the displacement mechanism 62 is configured to displace the receiving antenna 53, but it may be configured to displace the transmitting antenna 52. In addition, the displacement mechanism 62 may be configured to displace both the receiving antenna 53 and the transmitting antenna 52. In other words, the displacement mechanism 62 only needs to be configured to displace at least one of the receiving antenna 53 or the transmitting antenna 52. Furthermore, the control data 65 only needs to be data indicating the relationship between the displacement amount of at least one of the receiving antenna 53 or the transmitting antenna 52 for one period and the rotational position.

Further, in the above embodiment, the displacement controller 63 is provided inside the stationary portion 2C, but the displacement controller 63 may be provided outside the stationary portion 2C, such as inside the console 4. As the hardware structure of the displacement controller 63, various processors shown below can be used. The various processors include, as described above, a CPU, which is a general-purpose processor that executes software (programs) and functions as various processing units, a programmable logic device (PLD), which is a processor of which the circuit configuration can be changed after manufacture, such as a field programmable gate array (FPGA), a dedicated electrical circuit as a processor having a dedicated circuit configuration for executing specific processing such as an application-specific integrated circuit (ASIC), and the like.

The displacement controller 63 may be configured by one of the various processors, or may be configured by a combination of the same or different kinds of two or more processors (for example, a combination of a plurality of FPGAs or a combination of the CPU and the FPGA).

From the above description, the technology described in the following supplementary notes can be understood.

### Supplementary Note 1

A CT apparatus comprising:
an X-ray source configured to radiate X-rays to a subject;
a detector configured to detect X-rays transmitted through the subject and output detection data;
a rotating portion configured to support the X-ray source and the detector and rotate around a rotation axis;
a stationary portion configured to rotatably hold the rotating portion;
a transmitting antenna provided on the rotating portion for transmitting the detection data;
a receiving antenna disposed at a position facing a part of the transmitting antenna;
a rotational position detection unit configured to detect a rotational position of the rotating portion and output a detection value;
a measurement unit configured to measure a relative relationship between the transmitting antenna and the receiving antenna and output a measurement value;
a displacement mechanism configured to displace at least one of the receiving antenna or the transmitting antenna; and
a processor configured to create control data based on the detection value and the measurement value, and execute displacement control to suppress a change in the relative relationship by controlling the displacement mechanism based on the control data during transmission and reception of the detection data.

### Supplementary Note 2

The CT apparatus according to Supplementary Note 1,
in which the processor is configured to create relative relationship data indicating a relationship between the relative relationship and the rotational position for one period based on the detection value and the measurement value for one period of rotation of the rotating portion, and create the control data based on the relative relationship data.

### Supplementary Note 3

The CT apparatus according to Supplementary Note 2,
in which the control data indicates a relationship between a displacement amount of at least one of the receiving antenna or the transmitting antenna for one period and the rotational position.

### Supplementary Note 4

The CT apparatus according to any one of Supplementary Notes 1 to 3,
in which the transmitting antenna is disposed along an outer peripheral portion or an inner peripheral portion of the rotating portion.

### Supplementary Note 5

The CT apparatus according to Supplementary Note 4,
in which the relative relationship is a distance between the transmitting antenna and the receiving antenna, and
the displacement mechanism is configured to displace the receiving antenna in a direction parallel to the rotation axis.

### Supplementary Note 6

The CT apparatus according to Supplementary Note 4,
in which the relative relationship is a parallelism between the transmitting antenna and the receiving antenna, and
the displacement mechanism is configured to displace the receiving antenna around an axis parallel to the rotation axis.

### Supplementary Note 7

The CT apparatus according to Supplementary Note 4,
in which the relative relationship is an overlap rate between the transmitting antenna and the receiving antenna, and
the displacement mechanism is configured to displace the receiving antenna around an axis parallel to a direction orthogonal to the rotation axis.

### Supplementary Note 8

The CT apparatus according to any one of Supplementary Notes 5 to 7, further comprising:
a receiver configured to receive the detection data via the receiving antenna,
in which the displacement mechanism is configured to displace the receiving antenna by displacing the receiver.

### Supplementary Note 9

The CT apparatus according to any one of Supplementary Notes 1 to 8,
in which the processor is configured to correct the control data based on the measurement value during execution of the displacement control.

### Supplementary Note 10

The CT apparatus according to Supplementary Note 9,
in which the processor is configured to correct the control data based on a difference value between the measurement value before one period, before a certain time, or before a certain rotation angle and the acquired measurement value.

What is claimed is:

1. A CT apparatus comprising:
an X-ray source configured to radiate X-rays to a subject;
a detector configured to detect X-rays transmitted through the subject and output detection data;
a rotating portion configured to support the X-ray source and the detector and rotate around a rotation axis;
a stationary portion configured to rotatably hold the rotating portion;
a transmitting antenna provided on the rotating portion for transmitting the detection data;
a receiving antenna disposed at a position facing a part of the transmitting antenna;
a rotational position detector configured to detect a rotational position of the rotating portion and output a detection value;
a measurement sensor configured to measure a relative relationship between the transmitting antenna and the receiving antenna and output a measurement value;
a displacement mechanism configured to displace at least one of the receiving antenna or the transmitting antenna; and
a processor configured to create control data based on the detection value and the measurement value, and execute displacement control to suppress a change in the relative relationship by controlling the displacement mechanism based on the control data during transmission and reception of the detection data.

2. The CT apparatus according to claim 1,
wherein the processor is configured to create relative relationship data indicating a relationship between the relative relationship and the rotational position for one period based on the detection value and the measurement value for one period of rotation of the rotating portion, and create the control data based on the relative relationship data.

3. The CT apparatus according to claim 2,
wherein the control data indicates a relationship between a displacement amount of at least one of the receiving antenna or the transmitting antenna for one period and the rotational position.

4. The CT apparatus according to claim 1,
wherein the transmitting antenna is disposed along an outer peripheral portion or an inner peripheral portion of the rotating portion.

5. The CT apparatus according to claim 4,
wherein the relative relationship is a distance between the transmitting antenna and the receiving antenna, and
the displacement mechanism is configured to displace the receiving antenna in a direction parallel to the rotation axis.

6. The CT apparatus according to claim 4,
wherein the relative relationship is a parallelism between the transmitting antenna and the receiving antenna, and
the displacement mechanism is configured to displace the receiving antenna around an axis parallel to the rotation axis.

7. The CT apparatus according to claim 4,
wherein the relative relationship is an overlap rate between the transmitting antenna and the receiving antenna, and
the displacement mechanism is configured to displace the receiving antenna around an axis parallel to a direction orthogonal to the rotation axis.

8. The CT apparatus according to claim 5, further comprising:
a receiver configured to receive the detection data via the receiving antenna,
wherein the displacement mechanism is configured to displace the receiving antenna by displacing the receiver.

9. The CT apparatus according to claim 1,
wherein the processor is configured to correct the control data based on the measurement value during execution of the displacement control.

10. The CT apparatus according to claim 9,
wherein the processor is configured to correct the control data based on a difference value between the measurement value before one period, before a certain time, or before a certain rotation angle and the acquired measurement value.

11. A control method for a CT apparatus including
an X-ray source configured to radiate X-rays to a subject,
a detector configured to detect X-rays transmitted through the subject and output detection data,
a rotating portion configured to support the X-ray source and the detector and rotate around a rotation axis,
a stationary portion configured to rotatably hold the rotating portion,
a transmitting antenna provided on the rotating portion for transmitting the detection data;
a receiving antenna disposed at a position facing a part of the transmitting antenna,
a rotational position detector configured to detect a rotational position of the rotating portion and output a detection value,
a measurement sensor configured to measure a relative relationship between the transmitting antenna and the receiving antenna and output a measurement value, and
a displacement mechanism configured to displace at least one of the receiving antenna or the transmitting antenna, the control method comprising:
creating, by a processor, control data based on the detection value and the measurement value; and
executing displacement control to suppress a change in the relative relationship by controlling the displacement mechanism based on the control data during transmission and reception of the detection data.
